(19) United States
(12) Patent Application Publication
(10) Pub. No.: US 2009/0177225 A1
(43) Pub. Date: Jul. 9, 2009

(54) Vascular closure device having sensor

(76) Inventors: Anthony Nunez, Beechwood, OH (US); Harry D. Rowland, East Peoria, IL (US)

Correspondence Address: Patent Docket Department;Armstrong Teasdale LLP, One Metropolitan Square, Suite 2600, St. Louis, MO, 63102-2740, US

(21) Appl. No.: 12/011,502

(22) Filed: Jan. 25, 2008

Related U.S. Application Data
(60) Provisional application No. 60/897,754, filed on Jan. 26, 2007.

(51) Int. Cl. A61B 17/08; A61B 5/0215

(52) U.S. Cl. 606/213; 600/486

(57) ABSTRACT

A vascular closure device including a first retainer positioned on an internal surface of a vessel wall defining a passage through a vessel. A second retainer is coupled to the first retainer and positioned on an outer surface of the vessel wall to seal an opening defined through the vessel wall. A sensor is coupled to the first retainer and configured to sense a physical, chemical, and/or physiological parameter within the passage.

### CROSS REFERENCE TO RELATED APPLICATIONS

This application claims the benefit of U.S. Provisional Application No. 60/897,754, filed Jan. 26, 2007, which is hereby incorporated by reference in its entirety.

### BACKGROUND OF THE INVENTION

This disclosure relates generally to a device that is incorporated into a vascular closure device and, more particularly, to a vascular closure device including a sensor configured to sense physical, chemical, and/or physiological parameters, such as a fluid pressure within a vessel.

The medical industry has been performing an increasing number of minimally invasive procedures for therapeutic and diagnostic purposes. These procedures usually involve the use of a catheter system including a catheter that is inserted through a vessel, such as an artery, to access problem areas in the body. These procedures require puncturing or otherwise penetrating the vessel wall to treat blood clots, aneurysms and other vascular defects or diseases. Some common procedures include angioplasty and stent grafting to address blood clot formation and aneurysms, respectively. These less invasive techniques allow the patient to enjoy shorter recovery times and less risk of infection and other disease associated with conventional surgery.

In an effort to ease these procedures simple vascular sealing devices have been developed to aid in the clotting and tissue rebuilding of the body while no longer needing the external attention of a nurse or physician. At least one of these vascular sealing devices utilizes retainers to seal the puncture defined through the vessel. The materials are commonly fabricated out of bioabsorbable materials allowing them to be left in the patient for an extended period of time, being absorbed by the body after the wound site has healed. During more conventional procedures, pressure is applied to the affected area of the patient in an attempt to decrease the blood flow to allow hemostasis and tissue rebuilding to take place before an excessive degree of hematoma has occurred.

### BRIEF DESCRIPTION OF THE INVENTION

In one aspect, a vascular closure device is provided. The vascular closure device includes a first retainer positioned on an internal surface of a vessel wall defining a passage through a vessel. A second retainer is coupled to the first retainer and positioned on an outer surface of the vessel wall to seal an opening defined through the vessel wall. A sensor is coupled to the first retainer and configured to sense at least one of a physical, chemical, and physiological parameter within the passage.

In another aspect, a vascular closure device is provided. The vascular closure device includes an inner retainer positioned on an internal surface of a vessel wall defining a passage through a vessel. The inner retainer includes a wireless sensor configured to sense at least one of a physical, chemical, and physiological parameter within the passage. An outer retainer is coupled to the inner retainer and positioned on an outer surface of the vessel wall to seal an opening defined through the vessel wall.

In another aspect, a method is provided for treating cardiovascular disease. The method includes positioning a sensor within a passage defined by a vessel wall. The sensor is configured to generate a signal representative of at least one of a physical, chemical, and physiological parameter within a vascular system of a patient. The signal is communicated to a patient signaling device located at least partially externally to the patient. The patient signaling device processes the signal to generate at least one instructive treatment signal to facilitate determining a pharmaceutical therapy.

In another aspect, a vascular closure device is provided that includes at least one sensor configured for measuring at least one of a physical, chemical, and physiological parameter of a patient.

In another aspect, a method is provided for measuring at least one of a physical, chemical, and physiological parameter of a patient. The method includes placing a permanent implant with respect to a vessel wall.

In another aspect, a device is provided for measuring at least one of a physical, chemical, and physiological parameter of a patient including a sensor permanently implanted in proximity to a vessel wall.

In another aspect, a single-piece flexible vascular closure device is provided. The single-piece flexible vascular closure device includes a first portion positioned within a vessel at an interface of the vascular closure device and a flow of blood through the vessel wall. A second portion transitions into the first portion. At a transition area, at least one of the first portion and the second portion forms a groove configured to receive a portion of a vessel wall defining an opening through the vessel. The second portion is positioned on an outer surface of the vessel wall to seal the opening. A sensor is operatively coupled to one of the inner portion and the outer portion. The sensor is configured to sense at least one of a physical, chemical, and physiological parameter within the vessel.

In another aspect, an implantable monitoring device is provided. The implantable monitoring device is positionable externally about a vessel and includes at least one load cell or sensor configured to sense at least one of a physical, chemical, and physiological parameter within the vessel.

In another aspect, an implantable device is provided. The implantable device includes a first material defining a void on a first surface. An electrically conducting surface is patterned in the first surface within the void. A plate is coupled to the first surface to enclose the void and form a hermetically sealed cavity. The plate is patterned with electrically functional components. The implantable device has pressure sensing capabilities.

### BRIEF DESCRIPTION OF THE DRAWINGS

FIG. 1 is a cross-sectional view of a vessel and an exemplary vascular closure device positioned at a puncture site on a vessel wall;

FIG. 2 is a cross-sectional view of a vessel and an alternative exemplary vascular closure device positioned at a puncture site on a vessel wall;

FIG. 3 is a perspective view of a pressure sensor coupled to a first retainer;

FIG. 4 is a top view of the vascular closure device shown in FIG. 1;

FIG. 5 is a side view of the vascular closure device shown in FIG. 1;

FIG. 6 is a perspective view of the vascular closure device shown in FIG. 1;

FIG. 7 is a schematic view of an alternative exemplary vascular closure device including an inner retainer configured with sensing capabilities;

FIG. 8 is a cross-sectional view of the inner retainer shown in FIG. 7;

FIG. 9 is a schematic view of an exemplary vascular closure device in electrical communication with a patch applied to a skin surface of a patient;

FIG. 10 is a schematic view of an exemplary delivery device suitable for use in implanting a vascular closure device within a vessel;

FIG. 11 is a schematic view of the delivery device shown in FIG. 10 with an inner retainer of the vascular closure device implanted within the vessel;

FIG. 12 is a schematic view of an exemplary outer retainer of the vascular closure device shown in FIGS. 10 and 11; and

FIG. 13 is a schematic view of an exemplary inner retainer of the vascular closure device shown in FIGS. 10 and 11;

FIG. 14 is a schematic view of an alternative exemplary vascular closure device;

FIG. 15 is a schematic view of the vascular closure device shown in FIG. 14 during deployment;

FIG. 16 is a schematic view of the vascular closure device shown in FIG. 14 deployed within an opening defined through a vessel wall;

FIG. 17 is a schematic view of a sensor coupled to a suture;

FIG. 18 is a schematic view of a sensor coupled to a suture, with the suture closing an opening defined within a vessel wall;

FIG. 19 is a schematic view of an exemplary implantable monitoring device; and

FIG. 20 is a schematic view of an incision exposing a vessel passage and the implantable monitoring device shown in FIG. 19 positionable about the vessel with the incision closed.

### DETAILED DESCRIPTION OF THE INVENTION

The present disclosure describes exemplary embodiments of a permanently, semi-permanently or temporarily implanted device, such as a vascular closure device, that includes a sensor for measuring one or more physical, chemical, and/or physiological parameters or variables of a patient including, without limitation, a pressure, a temperature, and/or a cardiac output, for example. The device is implanted with respect to a vessel wall for measuring at least one of a physical, chemical, and physiological parameter or variable of a patient. In alternative embodiments, the vascular closure device may include any suitable coupling mechanism and/or anchoring mechanism including, without limitation, one or more retaining members, one or more clips, and/or one or more sutures, as described herein for example. Further, one or more sensors may be positioned within the vessel or may be positioned extravascular and with respect to the vessel wall. In a particular embodiment, at least one sensor is implanted and positioned at or near an inner surface of the vessel wall and/or at or near an outer surface of the vessel wall and configured to measure blood pressure. In a further embodiment, the vascular closure device is a single-piece flexible vascular closure device having sensing capabilities, as described in greater detail below.

The embodiments described herein provide a biomedical sensor, such as a pressure sensor, that is positioned within a vessel including, without limitation, a blood vessel such as an artery or a vein. In one embodiment, the sensor is coupled to or integrated with a vascular closure device. The sensor transmits sensed or detected readings wirelessly to an external system or a user, such as a doctor, a nurse or a medical technician, through RF signals without the requirements of an internal powering system. In a particular embodiment, the sensor is energized through electromagnetic fields that are directed to a circuitry of the sensor by the user.

In addition to sealing an opening defined within a vessel at a puncture site, additional diagnostic data, such as data representative of physical, chemical, and/or physiological parameters or variables, may be useful to the practicing physician to assist in management of patient care after the procedure. One such variable is the internal pressure of the vessel, commonly referred to as the blood pressure. For example, the practicing physician may utilize data, such as blood pressure data, to assist in directing patient care and making decisions regarding administration of blood pressure medication. Until recently, pressure sensors of the microscale were not available for common use. The recent development of microelectromechanical systems (MEMS) has enabled small functional systems to be manufactured on a large scale enabling low cost and high volume output of such products.

One such pressure sensor formed using a MEMS technique has an inductive and capacitive nature. The sensor acts as an inductor (L) and a capacitor (C) connected together in parallel, commonly called an LC tank circuit. The geometry of the sensor allows for the deformation of a capacitive plate with increased pressure. This deformation leads to a deflection of the plate and hence a change in the capacitance value of the system. The LC tank circuit also generates an electronic resonating frequency. This resonating frequency is related to the inductive and capacitance values of the circuit and will change with the deflection of capacitor plates under changing pressure. This emitted resonating frequency signal is received by an external wireless receiver and deciphered into a correlative pressure reading.

Such devices may also include wireless data transmission ability. The device requires no battery or internal power. Rather, the device is powered by an electromagnetic (EM) field that is directed towards the inductor coil. The inductor receives energy from the EM field to charge the capacitor, where the value of the capacitance varies with environmental pressure. When the EM field is removed, the inductance and capacitance form a parallel resonant circuit to radiate energy through the inductor which acts as an antenna. This oscillating circuit will then produce inherent radio frequency (RF) signals, which are proportional to the capacitive values of the sensor. The inductor coil serves both as an inductor creating the oscillating RF signals having a frequency proportional to the capacitance of the sensor at a certain pressure, and as an antenna coil emitting the RF signal generated by the LC tank circuitry.

The sensor is suitable for short-term use or long-term use. In one embodiment in which the sensor is configured as a long-term diagnostic tool for physicians, the sensor is coupled to or integrated with a radial inner retainer of the vascular closure device. In one embodiment, the inner retainer is coupled to an outer retainer through an anchoring member to prevent the vascular closure device from dislodging and becoming a free moving body in the patient's vascular system. In a particular embodiment, the outer retainer and/or the anchoring member is made from a non-absorbable biocompatible material. Alternatively, the outer retainer and/or the anchoring member are made from an absorbable or dissolvable biocompatible material.

In certain embodiments with the sensor serving as the inner retainer of a vascular closure device, the sensor contacts the internal surface of the vessel wall to seal or close the puncture or opening defined through the vessel wall. The contact between the sensor and the internal surface of the vessel wall promotes homeostasis at the puncture site. The geometric shape, design and/or dimensions of the sensor Facilitate reducing the risk of thrombosis or excessive cellular growth on the sensor. In one embodiment, the sensor is coated with one or more biocompatible materials. For example, a first or radial inner portion of the sensor is coated with an anti-metabolite or antithrombotic material that prohibits or limits the formation of thrombosis and/or potential embolization of vascular clots in the patient's vascular system, which may cause vascular occlusion to occur. The antimetabolite material may also promote tissue healing in a controlled manner so as to limit a degree of intimal growth while still encouraging a limited or minimum number of cell layers that independently provide an anti-thrombotic protective effect. A second or radial outer portion, which is adjacent an internal surface of the vessel wall, in contrast is coated in a material that promotes the formation of tissue growth about the inner retainer and/or the sensor to facilitate coupling the inner retainer and/or the sensor to the vessel wall for long-term diagnostic use.

Although the following disclosure describes a sensor that is configured to measure and/or monitor a blood pressure within the vessel to facilitate obtaining data for blood pressure analysis, it should be apparent to those skilled in the art and guided by the teachings herein provided that the sensor as described herein may be configured to measure physical, chemical, and/or physiological parameters or variables to facilitate obtaining data for temperature analysis, blood chemical analysis, blood osmolar analysis, and cellular count analysis, for example.

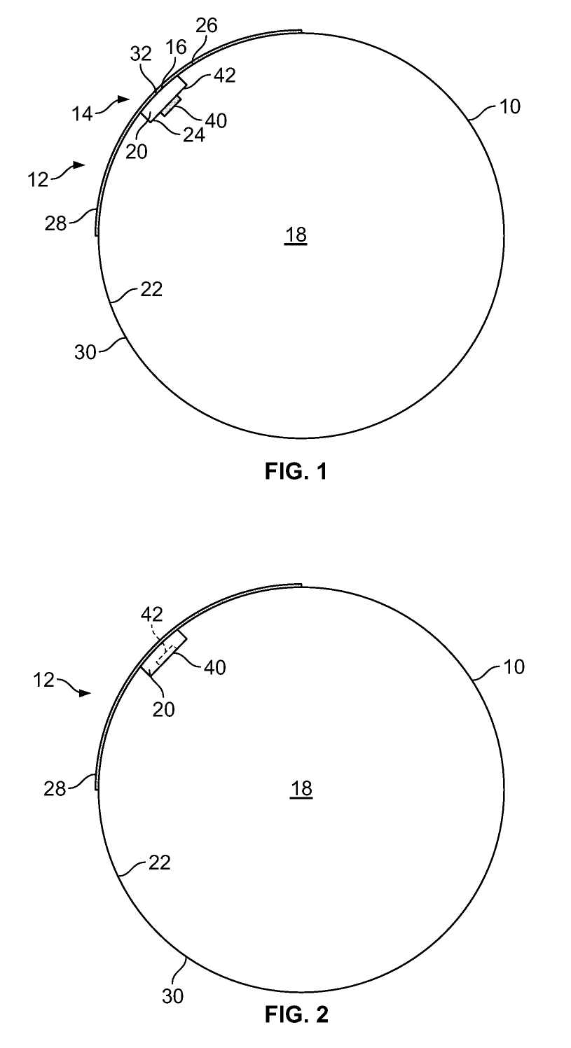

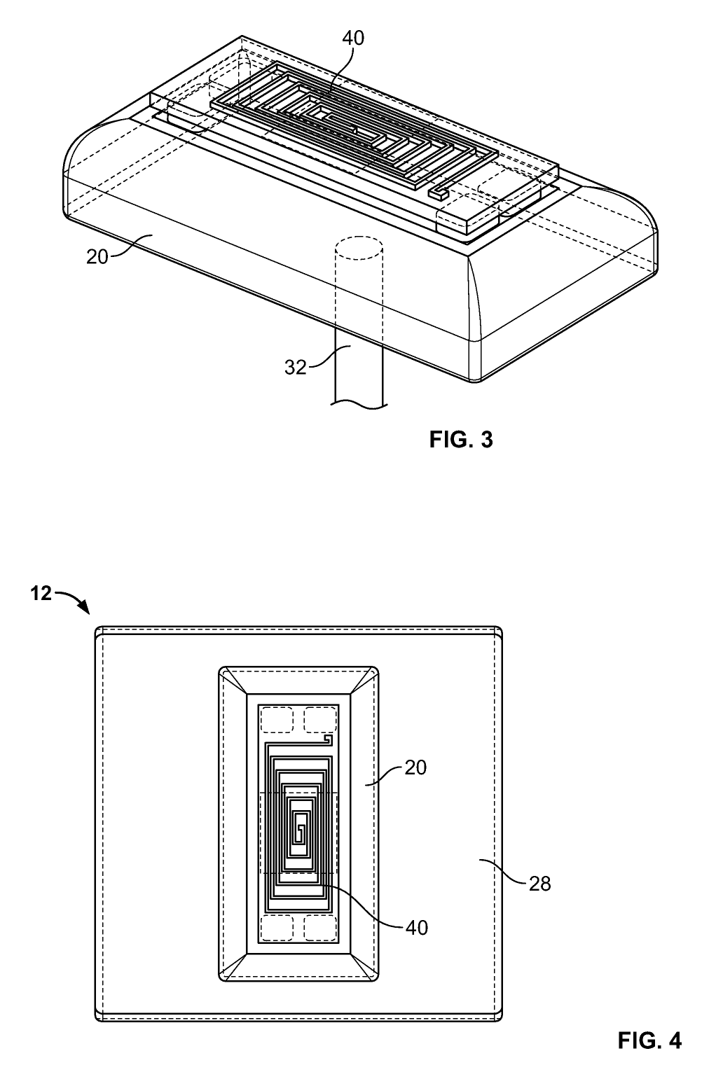

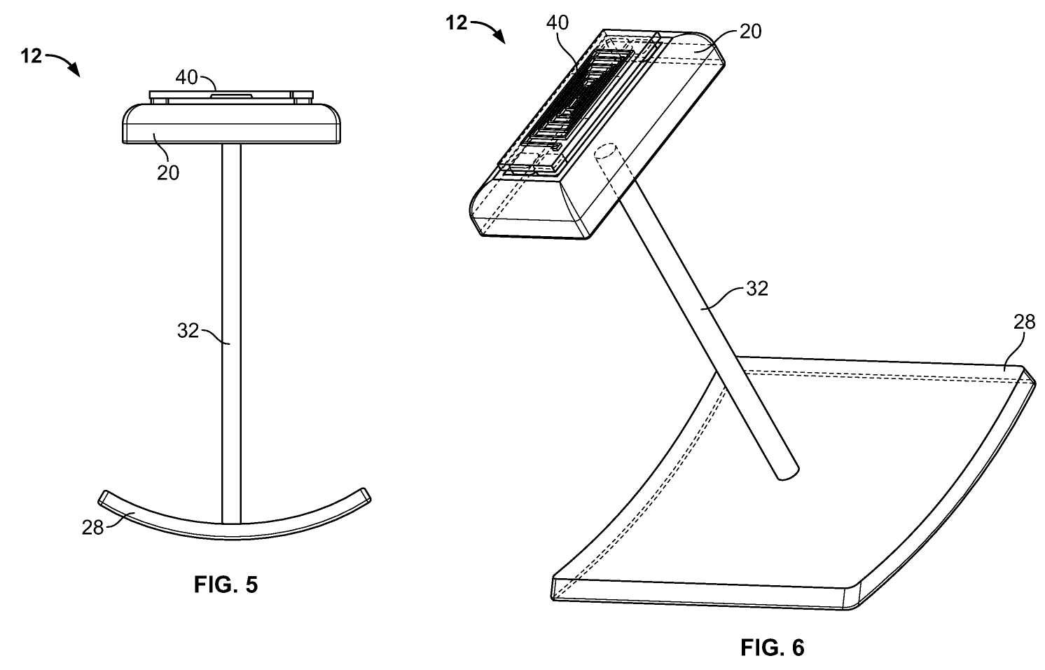

FIG. 1 is a cross-sectional view of a vessel wall 10 and an exemplary vascular closure device 12 coupled at or near a puncture site 14 on vessel wall 10. Vascular closure device 12 is configured to close and seal an opening 16, such as a puncture, piercing or other penetration, defined through vessel wall 10 at puncture site 14. For example, opening 16 may be formed by a defect or disease or may result from introduction of a needle or catheter through vessel wall 10 into a passage 18 defined by vessel wall 10. Vessel wall 10 forms passage 18 along a length of a vessel, such as an artery or a vein of a patient's cardiovascular system. Vascular closure device 12 is also configured with sensing capabilities.

Vascular closure device 12 includes a first or radial inner retainer 20 positioned on an internal surface 22 of vessel wall 10 at an interface of vascular closure device 12 and a flow of blood through the vessel. With inner retainer 20 positioned at or near internal surface 22, accurate measurements of laminar blood flow with limited fluctuation in measurement data are obtained, as described in greater detail herein. In contrast to vascular closure device 12, conventional internal pressure sensors are positioned at or near a center axis of the vessel and measure turbulent blood flow, resulting in less accurate measurement readings and/or greater fluctuation in measurement data. Additionally, when compared to conventional internal pressure sensors, the low profile of inner retainer 20 prevents or limits vessel passage occlusion.

Inner retainer 20 may have any suitable size and/or configuration. For example, inner retainer 20 may have an arcuate or curved cross-sectional profile. Alternatively, inner retainer 20 may have a generally planar configuration having a rectangular, square, star, oval, circular, or any other suitable polygonal or non-polygonal shape. Further, inner retainer 20 may have a helical shape. Inner retainer 20 may be fabricated of any suitable biocompatible material including, without limitation, an absorbable or non-absorbable material, such as non-absorbable prolene, a wholly degradable, partially degradable or non-degradable material, and/or a flexible material with a relatively small modulus of elasticity or a rigid material with a relatively large modulus of elasticity to facilitate permanent placement of a sensor with respect to passage 18, as described in greater detail herein. Alternatively or in addition, inner retainer 20 may be fabricated using a radio-opaque material, such as a barium sulfate material, to facilitate fluoroscopic visualization for future re-intervention, for example. In a further embodiment, inner retainer 20 is fabricated using a MRI-compatible material.

In one embodiment, a radially inner surface 24 of inner retainer 20 is coated with one or more materials that promotes tissue healing in a controlled manner. One or more coating layers may limit a degree of intimal growth while still encouraging a limited or minimum number of cell layers that provide an anti-thrombotic protective effect. The coating on inner surface 24 facilitates controlling the amount of cell or tissue growth over inner surface 24. Control of tissue growth facilitates limiting an amount of tissue growth, which may promote vessel occlusion, to promote wound healing while still enabling unobstructed blood flow through the vessel passage. Further, smaller amounts of tissue growth may undesirably limit efficacy of the wound closure. However, smaller amounts of tissue growth on inner surface 24 may improve the accuracy of sensor 40. Suitable coatings include, without limitation, materials such as materials that induce a negative or positive charge on the surface of the material, antimetabolites, heparin bonded ePTFE, a slow release polymer agent, a drug eluting material, such as Tacrolimus or Sirolimus, and/or another suitable antimetabolite. Further, a radially outer surface 26 of inner retainer 20 may be coated with a material that promotes formation of tissue growth on internal surface 22 of vessel wall 10. Such coating may promote hemostasis, improve adhesion of inner retainer 20 to vessel wall 10, and/or enable inner retainer 20 to be permanently affixed to vessel wall 10. Additionally, one or more surfaces, such as radially inner surface 24 and/or radially outer surface 26, of inner retainer 20 may be patterned with features, such as patterned or arbitrarily positioned projections and/or undulations, ranging in size from about 10 nanometers (nm) to about 100 micrometers (μm). The features may be used independently or in conjunction with chemical coatings to promote or inhibit tissue growth and/or modify the characteristics of blood flow over one or more surfaces of inner retainer 20. Each surface of inner retainer 20 may be flat, curved or may have any arbitrary three-dimensional shape.

A second or radial outer retainer 28 is coupled to inner retainer 20 and positioned on an outer surface 30 of vessel wall 10 to seal opening 16 defined through vessel wall 10. An anchoring member 32, such as a post or a pin, is positioned within opening 16 and couples inner retainer 20 to outer retainer 28. In one embodiment, anchoring member 32 is held in minimal tension creating compressive forces between inner retainer 20 and outer retainer 28, which apply a compression force on vessel wall 10 at or near puncture site 14 to subsequently seal opening 16. More specifically, compressive forces between inner retainer 20 and outer retainer 28 urge and retain radial outer surface 26 of inner retainer 20 in contact with internal surface 22 of vessel wall 10 and a radial inner surface 34 of outer retainer 28 in contact with outer surface 30 of vessel wall 10. In a particular embodiment, radial outer surface 26 and/or radial inner surface 34 has an arcuate cross-sectional profile to correspond with a cross-sectional profile of vessel wall 10. In at least certain biomedical applications, the compressive force on vessel wall 10 promotes homeostasis at puncture site 14 to facilitate securing vascular closure device 12 properly positioned at puncture site 14 and/or to facilitate patient recovery.

Outer retainer 28 may have any suitable size and/or configuration, including any size and/or configuration as described above in reference to inner retainer 20. As shown in FIGS. 1 and 2, outer retainer 28 has dimensions. such as a width or a diameter, greater than corresponding dimensions of inner retainer 20 to prevent vascular closure device 12 from undesirably entering into the vessel and/or to enable vascular closure device 12 to seal opening 16 and achieve hemostasis. Further, outer retainer 28 and/or anchoring member 32 may be fabricated of any suitable biocompatible material, including any material as described above in reference to inner retainer 20. For example, outer retainer 28 and/or anchoring member 32 may be fabricated using an absorbable or non-absorbable material, a wholly degradable, partially degradable or non-degradable material, and/or a flexible material with a relatively small modulus of elasticity or a rigid material with a relatively large modulus of elasticity. Alternatively or in addition, outer retainer 28 may be fabricated using a radio-opaque material, such as a barium sulfate material, to facilitate fluoroscopic visualization for any future re-intervention, for example. In further embodiments, outer retainer 28 is fabricated using a MRI-compatible material.

Further, outer retainer 28 may be coated with one or more coating layers of at least one biocompatible, absorbable or non-absorbable material that promotes cell growth and tissue healing to aid in wound closure. In one embodiment, at least one coating material promotes formation of tissue growth on outer surface 30 of vessel wall 10. Tissue growth enables outer retainer 28 to be permanently or semi-permanently affixed in the subcutaneous tissue at outer surface 30 of vessel wall 10. Using biocompatible materials to fabricate outer retainer 28 and/or the coating layers on outer retainer 28 may promote hemostasis and/or tissue in-growth. Additionally, one or more layers of a material, such as collagen, may be applied to radially inner surface 34 and/or a radially outer surface 36 of outer retainer 28 to promote tissue in-growth and/or wound closure. In one embodiment, outer retainer 28 contains active or passive circuitry with electrical functionality. For example, outer retainer 28 may contain a power source, such as a battery, an antenna, and/or an integrated circuit chip. Such circuitry processes, stabilizes, and/or boosts signals received from sensing elements in vascular closure device 12.

As described above, anchoring member 32 may be fabricated of any suitable biocompatible material, including any material as described above in reference to inner retainer 20. For example, anchoring member 32 may be fabricated using an absorbable or non-absorbable material, a wholly degradable, partially degradable or non-degradable material, and/or a flexible material with a relatively small modulus of elasticity or a rigid material with a relatively large modulus of elasticity to facilitate permanent placement of a sensor. Alternatively or in addition, anchoring member 32 may be fabricated using a radio-opaque material, such as a barium sulfate material, to facilitate fluoroscopic visualization for any future re-intervention for example. In further embodiments, anchoring member 32 is fabricated using a MRI-compatible material.

In one embodiment, anchoring member 32 is fabricated of a wire material including, without limitation, a suitable metal, alloy or composite material, such as nickel-titanium alloy, stainless steel, cobalt-based alloy, tantalum, gold, platinum or platinum-iridium for enhanced radio-opacity. In a particular embodiment, anchoring member 32 is coated with at least one coating material that promotes formation of tissue growth on vessel wall 10.

In one embodiment, anchoring member 32 is at least partially fabricated of a conducting material to provide electrical communication between inner retainer 20 and outer retainer 28. Anchoring member 32 may extend through, around, or into inner retainer 20 and/or outer retainer 28. Anchoring member 32 is coupled to inner retainer 20 and outer retainer 28 using any suitable attachment mechanism known to those skilled in the art and guided by the teachings herein provided, such as suturing, melting, adhesive bonding, and/or soldering. In one embodiment, anchoring member 32 is fabricated using a rigid tube or shaft. Alternatively, anchoring member 32 is fabricated using a flexible or bendable tube, thread or rope.

In an exemplary embodiment, one or more sensors 40 are operatively coupled, directly or indirectly, to or incorporated within vascular closure device 12. Referring further to FIGS. 1 and 2, in one embodiment, one or more sensors 40 are operatively coupled to inner retainer 20 and configured to sense a physical, chemical, and/or physiological parameter or variable including, without limitation, a blood pressure within the vessel. Additionally or alternatively, one or more sensors 40 are operatively coupled to outer retainer 28 and configured to sense a physical, chemical, and/or physiological parameter or variable including, without limitation, a blood pressure within the vessel. In an alternative embodiment, sensor 40 is operatively coupled to vascular closure device 12 without being directly coupled to inner retainer 20. In a particular alternative embodiment, sensor 40 is coupled on inner retainer 20 during insertion into the vessel and subsequently attaches to vessel wall 10 after the vascular closure device 12 has been successfully implanted. Sensor 40 may be a pressure sensor, an optical sensor, a biochemical sensor, a protein sensor, a motion sensor (e.g., an accelerometer or a gyroscope), a temperature sensor, a chemical sensor (e.g., a pH sensor), or a genetic sensor, for example.

In particular embodiments, sensor 40 includes a piezoelectric pressure sensing device, a capacitive pressure sensing device, or a piezoresistive pressure sensing device. In one embodiment, sensor 40 is coupled to radial inner surface 24 of inner retainer 20, as shown in FIG. 1, and positioned at the vascular closure device/blood flow interface. In an alternative embodiment, sensor 40 is integrated with inner retainer 20, as shown in FIG. 2, such that sensor 40 is capable of providing high fidelity readings or measurements of intravascular pressure, for example. More specifically, a void or well 42 is formed in inner surface 24 of inner retainer 20 having a shape and configuration corresponding to a shape and configuration of sensor 40 to facilitate integrating sensor 40 with inner retainer 20. In a further alternative embodiment, inner retainer 20 is a sensor having sensing capabilities, as described in greater detail below.

In one embodiment, sensor 40 has dimensions smaller than corresponding dimensions of radially inner surface 24 of inner retainer 20. In a particular embodiment, sensor 40 has overall dimensions of less than about 3 millimeters (mm) by 10 mm, while having a thickness of less than about 1 mm. It should be apparent to those skilled in the art and guided by the teachings herein provided, that sensor 40 may have any suitable dimensions such that vascular closure device 12 functions as described herein. As described above, sensor 40 has a suitable size and/or configuration such that laminar blood flow, rather than turbulent blood flow, is sensed and/or monitored.

In the embodiments shown in FIGS. 1 and 2, sensor 40 is coupled to inner retainer 20 using a suitable biocompatible material. In one embodiment, sensor 40 is coupled to inner retainer 20 using a suitable biocompatible adhesive including, without limitation, an acrylic-based adhesive, such as cyanoacrylate, an epoxy-based adhesive, a polyurethane-based adhesive, and/or a silicon-based adhesive, such as organopolysiloxane. Additionally or alternatively, sensor 40 is coupled to inner retainer 20 using a suitable mechanism and/or process known to those skilled in the art and guided by the teachings herein provided including, without limitation, a chemical bonding process, a heat bonding process, a soldering process, a suturing process using a non-absorbable suture, or an outer packaging material. In a particular embodiment, the outer packaging material is chemically treated with a suitable heparin-bonded ePTFE material, a drug eluting material that inhibits cellular overgrowth, such as Tacrolimus or Sirolimus, or another suitable anti-metabolite to provide an anti-thrombotic outer surface.

Sensor 40 is coupled to or integrated with inner retainer 20 during or after fabrication of inner retainer 20. In one embodiment, sensor 40 is coupled to inner retainer 20 using a suitable process to minimize restriction. obstruction, and/or occlusion of fluid flow through passage 18 at or near puncture site 14 with sensor 40 positioned within passage 18. In a particular embodiment, inner retainer 20, second retainer 28, and/or anchoring member 32 is fabricated of a suitable bioabsorbing or dissolving material such that, upon absorption or dissolution of inner retainer 20, outer retainer 28, and/or anchoring member 32, sensor 40 subsequently is permanently or semi-permanently attached to internal surface 22 of vessel wall 10.

In one embodiment, sensor 40 is fabricated using a suitable microelectromechanical systems (MEMS) technology. In a particular embodiment, sensor 40 is fabricated using a MEMS technology that utilizes a resonating frequency of an LC Tank circuit or a suitable capacitive or piezoelectric technology to measure pressure within passage 18. Sensor 40 is configured to facilitate transmission of data wirelessly to an external device, such as a user-controlled receiver. In a biomedical application, the signal is desirably transmitted through the patient's surrounding tissue without distorting or lowering a strength of the signal such that the signal is lost of undecipherable. In this embodiment, sensor 40 includes a capacitance inductor circuit arranged in a parallel configuration to form an LC tank circuit. The LC tank circuit generates resonating frequency signals that are emitted through the vessel and transmitted to an at least partially external device, such as a patient signaling device, wherein the signals are processed and deciphered to facilitate treating defects and/or disease, such as cardiovascular disease. The external device generates an output representative of an internal pressure of the vessel. More specifically, in one embodiment, sensor 40 is positioned within vessel 10 and configured to sense blood flow through vessel 10 to facilitate measuring and/or monitoring blood pressure, for example. Sensor 40 generates a signal representative of a fluid pressure within passage 18. It should be apparent to those skilled in the art and guided by the teachings herein provided that sensor 40 may be fabricated using any suitable technology and/or process in alternative embodiments.

In one embodiment, sensor 40 is coated with at least one biocompatible material including, without limitation, one or more suitable biocompatible polymers such as a slow release polymer impregnated with an anti-metabolite inhibiting in-tissue growth. In a particular embodiment, a first portion of sensor 40 is coated with a drug eluting material that prohibits in-tissue growth on sensor 40. A second portion of sensor 40 is coated with a material promoting the in-tissue growth on sensor 40 to affix sensor 40 to vessel wall 10 for long term use.

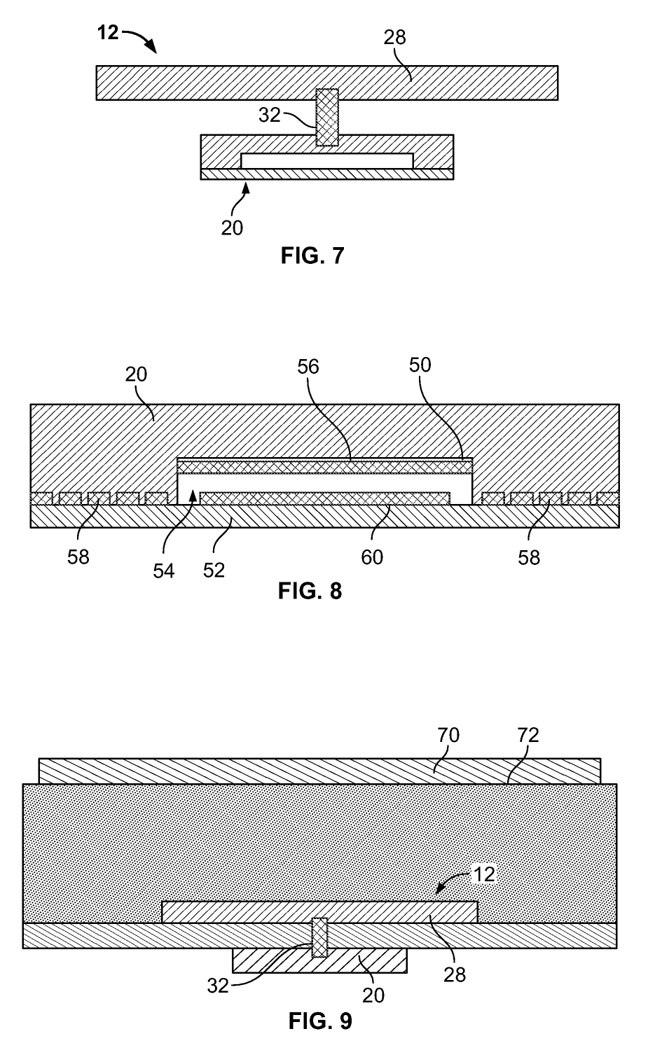

In one embodiment, inner retainer 20 may be configured with sensing capabilities, such as shown in FIGS. 7 and 8. In a particular embodiment, inner retainer 20 includes a wireless capacitive pressure sensor having pressure sensing capabilities. A void 50 is formed on radially inner surface 24 of inner retainer 20 and a plate or layer of material 52 is coupled to radially inner surface 24 to enclose void 50, thus forming a hermetically sealed cavity 54 in inner retainer 20. Plate 52 is fabricated of any suitable material including, without limitation, a polymer, silicon, or fused silica material. In a particular embodiment, plate 52 is patterned with electrically functional components, such as conductors, dielectrics, capacitors, resistors, and/or semiconductors. The components are patterned or deposited using suitable semiconductor fabrication techniques and/or other suitable printed electronics techniques known to those skilled in the art and guided by the teachings herein provided. In one embodiment, the components are formed by depositing a conducting seed layer, depositing a photoresist on the seed layer, patterning the photoresist, and electroplating components on the exposed regions of the seed layer. Additional portions of inner retainer 20 may also be patterned with electrically functional components. As shown in FIG. 8, an electrically conducting surface 56 is patterned in void 50 formed in radially inner surface 24 of inner retainer 20 and partially forming cavity 54. In one embodiment, electrically conducting surface 56 forms one surface of a capacitor plate.

In a particular embodiment, conducting components, such as an inductor component 58 including an antenna and/or a capacitor plate 60, are patterned on plate 52. Conducting components, such as electrically conducting surface 56, inductor 58, and capacitor plate 60 are operatively coupled to form an LC tank circuit. In one embodiment, inductor 58 and capacitor plate 60 are electrically coupled in a parallel circuit. Plate 52 is configured to deform when a pressure is applied to plate 52. The deformation produces a change in capacitance which causes a change in the resonant frequency of the LC tank circuit. The resonant frequency of the LC tank circuit may be monitored remotely and/or wirelessly with electronic components external to the body. One or more surfaces of inner retainer 20 may be structured and/or coated to control or limit tissue growth on inner retainer 20 to enable high fidelity wireless readings of intravascular pressure, for example.

As shown in FIG. 9, in one embodiment, a patch 70 is applied external to a skin surface 72 of the patient to facilitate wireless communication between an external electronic device (not shown) and the implanted sensor 40. In one embodiment, patch 70 includes suitable electronic components configured to communicate with sensor 40 and boost signal transmitted through the patient's skin and subcutaneous layer, such as signals transmitted to sensor 40 from the external device and signals transmitted from sensor 40 to the external device. In a particular embodiment, patch 70 includes passive and/or active circuitry. Patch 70 may be fabricated with printed electronic technology to enable a low-cost disposable device that wirelessly communicates with implanted sensor 40 and the external electronic device or system.

In one embodiment, a wire extension is provided to couple sensor 40 in signal communication with patch 70 or a booster unit positioned within a subcutaneous tissue of the patient or incorporated into or placed above a collagen plug. The wire is also utilized with a suitable vascular closure device delivery system to hold a footplate of inner retainer 20 in proper position at puncture site 14. The wire may be made of any suitable metal, alloy and/or composite material including, without limitation, a nickel-titanium alloy, stainless steel, cobalt-based alloy, tantalum, gold, platinum and/or platinum-iridium for enhanced radio-opacity.

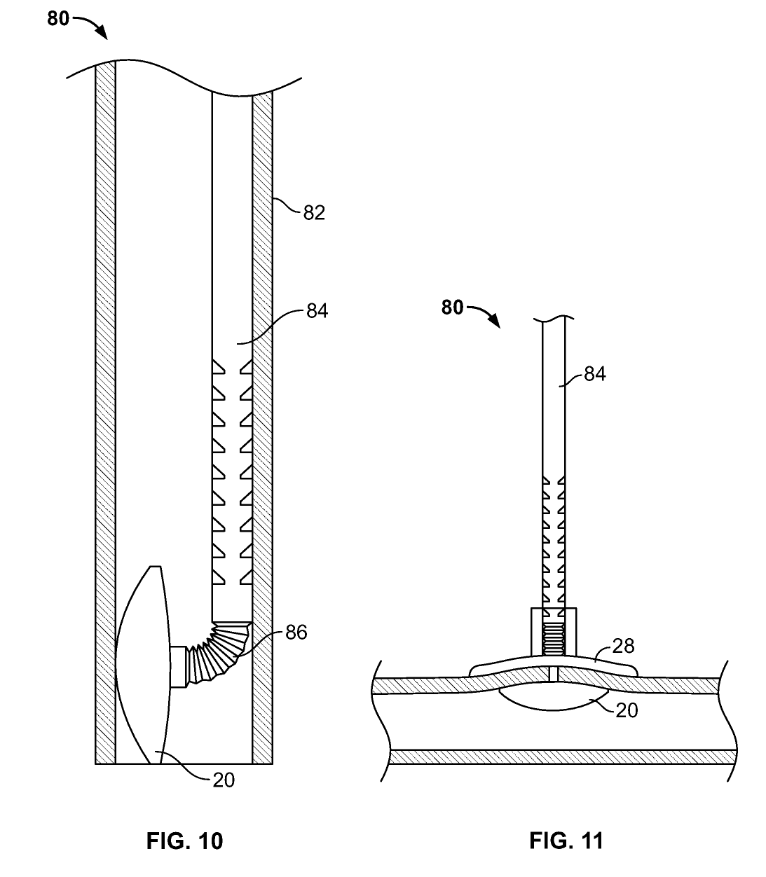

In one embodiment, vascular closure device 12 is deployed using a suitable catheter deliverable technology known to those skilled in the art and guided by the teachings herein provided. Referring to FIGS. 10 and 11, a delivery device 80 delivers and deploys inner retainer 20 within a vessel. Delivery device 80 includes a catheter delivery tube 82. An elongated anchoring mechanism 84 is connected to inner retainer 20 using a bendable, curved or corrugated packaging mechanism 86. In one embodiment, at least a portion of anchoring mechanism 84 is corrugated to allow positioning and/or bending to facilitate a minimally invasive delivery of inner retainer 20. In a particular embodiment, anchoring mechanism 84 is bendable by a suitable angle, such as at least about 90°, to facilitate delivery. Inner retainer 20 is advanced in catheter delivery tube 82 into the vessel. When an outer sheath of catheter delivery tube 82 is removed from about inner retainer 20, inner retainer 20 moves to an initial or deployed configuration. In an alternative embodiment, a bent, curved or corrugated packaging mechanism 86 is constructed of a material having shape memory characteristics, such as a polymer material, a metal alloy such as Nitinol, a spring-loaded mechanism, or another suitable material and/or mechanism.

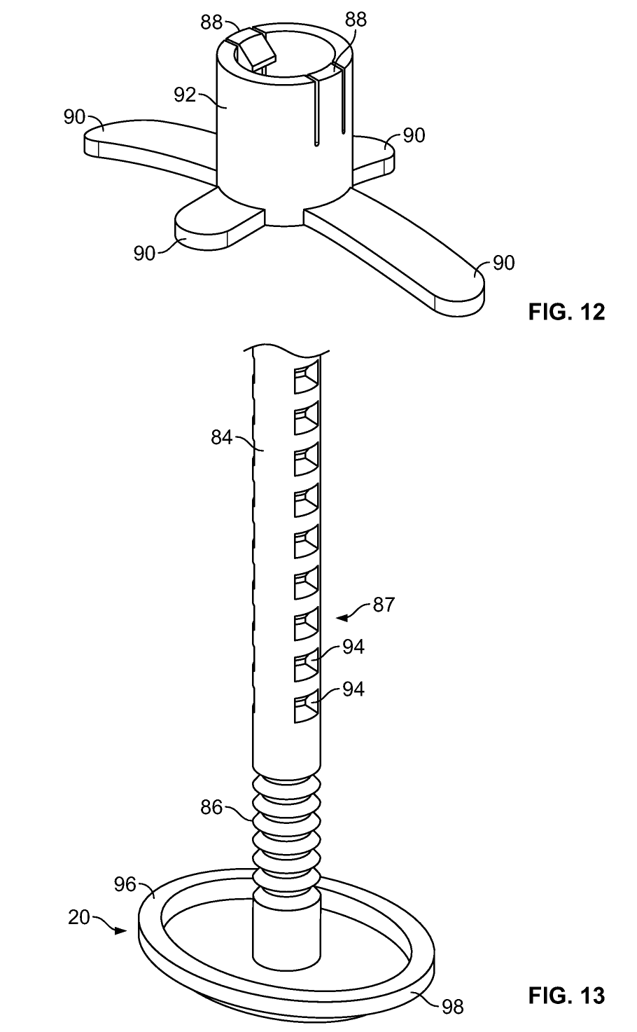

FIG. 11 schematically shows inner retainer 20 after delivery and in a deployed configuration within the vessel. Outer retainer 28 is guided down catheter delivery tube 82 over anchoring mechanism 84 or another suitable guidewire. In one embodiment, anchoring mechanism 84 includes a tab and ratchet mechanism 87 and outer retainer 28 includes a corresponding one-way ratchet 88, as shown in FIG. 12, to lock outer retainer 28 in position with tension in anchoring mechanism 84 retaining inner retainer 20 and outer retainer 28 coupled and pressed against vessel wall 10. A collagen plug or other suitable material may be inserted above or below outer retainer 28 to promote tissue healing and/or wound closure. FIG. 12 shows an exemplary embodiment of outer retainer 28 with branched structures 90 to aid in wound closure, a hollow shaft 92, and ratchets 88 formed on shaft 92.

FIG. 13 shows an exemplary embodiment of inner retainer 20 and anchoring mechanism 84. Anchoring mechanism 84 includes a bent, curved or corrugated packaging mechanism 86, and a corrugated section including tab and ratchet mechanism 87 having a plurality of voids or openings 94 configured to receive a corresponding ratchet 88 for securing inner retainer 20 and outer retainer 28 in proper position at puncture site 14. In one embodiment, inner retainer 20 may have a notched rim or lip 96 formed along at least a portion of a periphery of a base 98 of inner retainer 20 to enable inner retainer 20 to pull partially into vessel wall 10 or any tissue or calcium deposits adhered to vessel wall 10. In a particular embodiment, lip 96 facilitates coupling base 98 into calcium deposits in order to avoid undermining and/or to facilitate sealing against deposit. Further, base 98 may be coated with a surface, such as a suitable nanosurface, to prevent or limit calcium deposit and/or optimize fluid dynamics.

In an alternative embodiment, outer retainer 28 is not required for acute or chronic use. In this embodiment, inner retainer 20 includes barbs, hooks, and/or an adhesive material to secure inner retainer 20 to vessel wall 10. Alternatively, outer retainer 28 is partially or fully dissolved after a suitable period of time. In a particular embodiment, inner retainer 20 includes barbs, hooks, an adhesive material, and/or another suitable securing mechanism and outer retainer 28 also provides a secure attachment. Outer retainer 28 may or may not dissolve over a period of time.

Anchoring mechanism 84 may be non-absorbable, partially absorbable, or fully absorbable. In one embodiment, a portion of anchoring mechanism 84 at or near inner retainer 20 is not absorbable while a portion of anchoring mechanism 84 away from inner retainer 20 is absorbable. Referring to FIG. 11, the portion of anchoring mechanism 84 positioned between inner retainer 20 and outer retainer 28 is not absorbable while the portion of anchoring mechanism 84 positioned on an opposing side of outer retainer 28 is absorbable such that over time the outer portion of anchoring mechanism 84 needed for device delivery (but not needed for securing inner retainer 20 to outer retainer 28) is absorbed in the body.

In further embodiments, at least a portion of vascular closure device 12 is absorbable to regulate a lifetime of the implanted vascular closure device 12. Alternatively, vascular closure device is not absorbable. For example, inner retainer 20 may be fabricated of a degradable material that is coated with a second or additional degradable material. Upon deployment, the second degradable material coating protects electronic components on inner retainer 20 from the surrounding environment. The inner retainer electronics function with high fidelity while protected from the surrounding environment. After a period of time, the second degradable material is dissolved, and the degradable material of inner retainer 20 is then exposed to the surrounding environment. Eventually, inner retainer 20 is completely dissolved. Alternatively, a portion of inner retainer 20 is fabricated of a degradable material. For example, the inner retainer electronic components are fabricated of non-absorbable material while the remaining portions of inner retainer 20 are fabricated of an absorbable material. After a period of time, a footplate of inner retainer 20 is absorbed and only sensor 40 remains. In one embodiment wherein sensor 40 is attached to inner retainer 20, sensor 40 is fabricated of a non-absorbable material while inner retainer 20 is fabricated of an absorbable material.

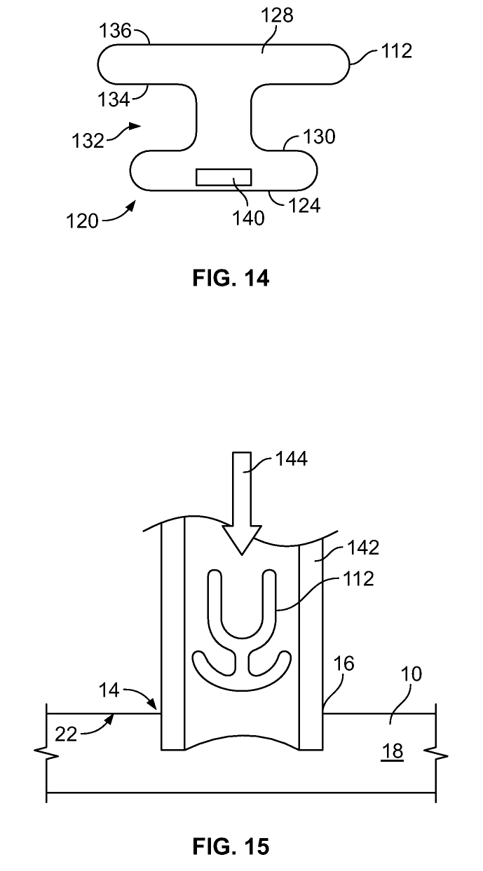

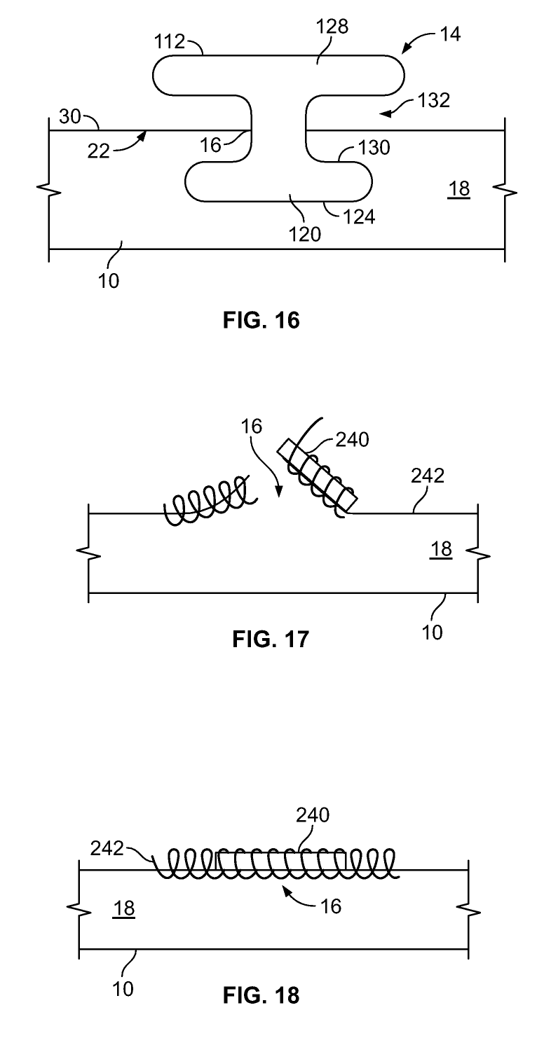

FIG. 14 is a schematic view of an alternative exemplary vascular closure device 112 that is configured to couple to a vessel wall 10 at or near a puncture site 14 on vessel wall 10. Referring further to FIGS. 15 and 16, vascular closure device 112 is configured to close and seal an opening 16, such as a puncture, piercing or other penetration, defined through vessel wall 10 at puncture site 14. For example, opening 16 may be formed by a defect or disease or may result from introduction of a needle or catheter through vessel wall 10 into a passage 18 defined by vessel wall 10. Vessel wall 10 forms passage 18 along a length of a vessel, such as an artery or a vein of a patient's cardiovascular system. Vascular closure device 112 is also configured with sensing capabilities.

In one embodiment, vascular closure device 112 is fabricated as a single piece device from a suitable biocompatible flexible material known to those skilled in the art and guided by the teachings herein provided. Further, vascular closure device 112 may be fabricated of any suitable biocompatible material including, without limitation, a non-absorbable material, Such as non-absorbable prolene, a non-degradable material, and/or a flexible material with a relatively small modulus of elasticity or a rigid material with a relatively large modulus of elasticity to facilitate permanent placement of a sensor with respect to passage 18, as described in greater detail herein. Alternatively or in addition, vascular closure device 112 may be fabricated using a radio-opaque material, such as a barium sulfate material, to facilitate fluoroscopic visualization for future re-intervention, for example. In a further embodiment, vascular closure device 112 is fabricated using a MRI-compatible material. Vascular closure device 112 may have any suitable size and/or configuration.

Vascular closure device 112 includes a first or radial inner portion 120 that is positioned the vessel, such as on an internal surface 22, shown in FIGS. 15 and 16, of vessel wall 10 at an interface of vascular closure device 112 and a flow of blood through the vessel. With inner portion 120 positioned at or near internal surface 22, accurate measurements of laminar blood flow with limited fluctuation in measurement data are obtained, as described in greater detail herein. In contrast to vascular closure device 112, conventional internal pressure sensors are positioned at or near a center axis of the vessel and measure turbulent blood flow, resulting in less accurate measurement readings and/or greater fluctuation in measurement data. Additionally, when compared to conventional internal pressure sensors, the low profile of inner portion 120 prevents or limits vessel passage occlusion.

In one embodiment, a radially inner surface 124 of inner portion 120 is coated with one or more materials that promote tissue healing in a controlled manner. One or more coating layers may limit a degree of intimal growth while still encouraging a limited or minimum number of cell layers that provide an anti-thrombotic protective effect. The coating on inner surface 124 facilitates controlling the amount of cell or tissue growth over inner surface 124. Control of tissue growth facilitates limiting an amount of tissue growth, which may promote vessel occlusion, to promote wound healing while still enabling unobstructed blood flow through the vessel passage. Further, smaller amounts of tissue growth may undesirably limit efficacy of the wound closure. Suitable coatings include, without limitation, materials such as materials that induce a negative or positive charge on the surface of the material, antimetabolites, heparin bonded ePTFE, a slow release polymer agent, a drug eluting material, such as Tacrolimus or Sirolimus, and/or another suitable antimetabolite. Further, a radially outer surface 130 of inner portion 120 may be coated with a material that promotes formation of tissue growth on internal surface 22 of vessel wall 10. Such coating may promote hemostasis, improve adhesion of inner portion 120 to vessel wall 10, and/or enable inner portion 120 to be permanently affixed to vessel wall 10. Additionally, one or more surfaces, such as radially inner surface 124 and/or radially outer surface 130, of inner portion 120 may be patterned with features, such as patterned or arbitrarily positioned projections and/or undulations, ranging in size from about 10 nanometers (nm) to about 100 micrometers (μm). The features may be used independently or in conjunction with chemical coatings to promote or inhibit tissue growth and/or modify the characteristics of blood flow over one or more surfaces of inner portion 120. Each surface of inner portion 120 may be flat, curved or may have any arbitrary three-dimensional shape.

Vascular closure device includes a second or radial outer portion 128 that is integrated with or transitions into to inner portion 120. At a transition area, inner portion 120 and/or outer portion 128 forms a suitable structure, such as a depression or groove 132, configured to receive a portion of vessel wall 10 defining opening 116, as shown in FIG. 16. Outer portion 128 is positioned on an outer surface 30 of vessel wall 10 to seal opening 16 defined through vessel wall 10.

Inner portion 120 and outer portion 128 may have any suitable size and/or configuration. As shown in FIG. 14, outer portion 128 has dimensions, such as a width or a diameter, greater than corresponding dimensions of inner portion 120 to prevent vascular closure device 112 from undesirably entering into the vessel and/or to enable vascular closure device 112 to seal opening 16 and achieve hemostasis.

Further, outer portion 128 may be coated with one or more coating layers of at least one biocompatible, absorbable or non-absorbable material that promotes cell growth and tissue healing to aid in wound closure. In one embodiment, at least one coating material promotes formation of tissue growth on outer surface 30 of vessel wall 10. Tissue growth enables outer portion 128 to be permanently or semi-permanently affixed in the subcutaneous tissue at outer surface 30 of vessel wall 10. Using biocompatible materials to fabricate outer portion 128 and/or the coating layers on outer portion 128 may promote hemostasis and/or tissue in-growth. Additionally, one or more layers of a material, such as collagen, may be applied to radially inner surface 134 and/or a radially outer surface 136 of outer portion 128 to promote tissue in-growth and/or wound closure. In one embodiment, outer portion 128 contains active or passive circuitry with electrical functionality. For example, outer portion 128 may contain a power source, such as a battery, an antenna, and/or an integrated circuit chip. Such circuitry processes, stabilizes, and/or boosts signals received from sensing elements in vascular closure device 112.

In an exemplary embodiment, one or more sensors 140 are operatively coupled, directly or indirectly, to or incorporated within vascular closure device 112. Referring further to FIG. 14, in one embodiment, one or more sensors 140 are operatively coupled to inner portion 120 and configured to sense one or more physical, chemical, and/or physiological parameters or variables including, without limitation, a blood pressure within the vessel. In an alternative embodiment, sensor 140 is operatively coupled to vascular closure device 112 without being directly coupled to inner portion 120. For example, sensors 140 may be operatively coupled, such as directly coupled to or integrated with outer portion 128 and configured to sense one or more physical, chemical, and/or physiological parameters or variables including, without limitation, a blood pressure within the vessel. Sensor 140 may be a pressure sensor, an optical sensor, a biochemical sensor, a protein sensor, a motion sensor (e.g., an accelerometer or a gyroscope), a temperature sensor, a chemical sensor (e.g., a pH sensor), or a genetic sensor, for example.

In particular embodiments, sensor 140 includes a piezoelectric pressure sensing device or a piezoresistive pressure sensing device. In one embodiment, sensor 140 is coupled to radial inner surface 124 of inner portion 120 and positioned at the vascular closure device/blood flow interface. In an alternative embodiment, sensor 140 is integrated with inner portion 120, as shown in FIG. 14, such that sensor 140 is capable of providing high fidelity readings or measurements of intravascular pressure, for example. In a further alternative embodiment, inner portion 120 is a sensor having sensing capabilities.

In one embodiment, sensor 140 is coupled to inner portion 120 using a suitable biocompatible material. In one embodiment, sensor 140 is coupled to inner portion 120 using a suitable biocompatible adhesive including, without limitation, an acrylic-based adhesive, such as cyanoacrylate, an epoxy-based adhesive, a polyurethane-based adhesive, and/or a silicon-based adhesive, such as organopolysiloxane. Additionally or alternatively, sensor 140 is coupled to inner portion 120 using a suitable mechanism and/or process known to those skilled in the art and guided by the teachings herein provided including, without limitation, a chemical bonding process, a heat bonding process, a soldering process, a suturing process using a non-absorbable suture, or an outer packaging material. In a particular embodiment, the outer packaging material is chemically treated with a suitable heparin-bonded ePTFE material, a drug eluting material that inhibits cellular overgrowth, such as Tacrolimus or Sirolimus, or another suitable anti-metabolite to provide an anti-thrombotic outer surface.

Sensor 140 is coupled to or integrated with inner portion 120 during or after fabrication of inner portion 120. In one embodiment, sensor 140 is coupled to inner portion 120 using a suitable process to minimize restriction, obstruction, and/or occlusion of fluid flow through passage 18 at or near puncture site 14 with sensor 140 positioned within passage 18. In one embodiment, sensor 40 is fabricated using a suitable microelectromechanical systems (MEMS) technology, such as described above in reference to sensor 40.

In one embodiment, sensor 140 is coated with at least one biocompatible material including, without limitation, one or more suitable biocompatible polymers such as a slow release polymer impregnated with an anti-metabolite inhibiting in-tissue growth. In a particular embodiment, a first portion of sensor 140 is coated with a drug eluting material that prohibits in-tissue growth on sensor 140. A second portion of sensor 140 is coated with a material promoting the in-tissue growth on sensor 140 to affix sensor 140 to vessel wall 10 for long term use.

Referring again to FIGS. 15 and 16, in one embodiment, flexible vascular closure device 112 is urged or pushed through a suitable catheter 142, as shown by arrow 144, in a compressed or folded configuration to opening 16, as shown in FIG. 15. With vascular closure device 112 positioned within opening 16, vascular closure device 112 moves to a deployed configuration, as shown in FIG. 16, wherein inner portion 120 is positioned within passage 18 and outer portion 128 is positioned on or against outer surface 30 of vessel wall 10. Vessel wall 10 is positioned within groove 132 to seal opening 16.

In an alternative embodiment as shown in FIGS. 17 and 18, a sensor 240 is coupled to a suture 242. In a particular embodiment, sensor 240 is inserted within suture loops and/or tied to suture 242. It should be apparent to those skilled in the art and guided by the teachings herein provided that any suitable process may be used for securing sensor 240 to suture 242. Suture 242 is used to close opening 16, as shown in FIG. 18. With opening 16 closed, sensor 240 may be positioned within passage 18 or sensor 240 may be positioned extravascular, such as shown in FIG. 18.

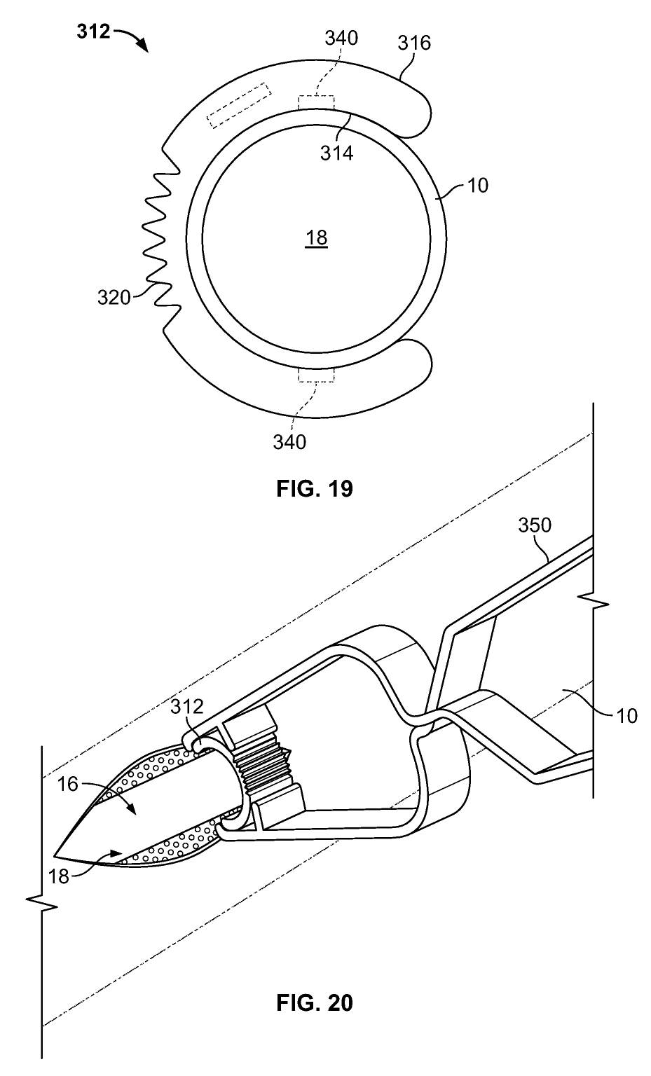

FIGS. 19 and 20 schematically show an exemplary implantable monitoring device 312 that is positionable about a vessel, such as a femoral artery, after closure of an incision in vessel wall 10 exposing passage 18. Referring further to FIGS. 19 and 20, implantable monitoring device 312 is configured to be placed externally or extravascularly about vessel wall 10 after opening 16, such as an incision, puncture, piercing or other penetration, defined through vessel wall 10. Implantable monitoring device 312 is also configured with sensing capabilities based, at least partially, on indirect expansion of the vessel.

In one embodiment, implantable monitoring device 312 is fabricated as a single-piece device from a suitable biocompatible material known to those skilled in the art and guided by the teachings herein provided. Implantable monitoring device 312 may also be fabricated using a radio-opaque material, such as a barium sulfate material, to facilitate fluoroscopic visualization for future re-intervention, for example. In a further embodiment, implantable monitoring device 312 is fabricated using a MRI-compatible material. Further, implantable monitoring device 312 may have any suitable size and/or configuration such that Implantable monitoring device 312 functions as described herein.

Implantable monitoring device 312 may be coated with one or more coating layers of at least one biocompatible, absorbable or non-absorbable material that promotes cell growth and tissue healing to aid in wound closure. In one embodiment, at least one coating material promotes formation of tissue growth on outer surface 30 of vessel wall 10. Tissue growth enables implantable monitoring device 312 to be permanently or semi-permanently affixed in the subcutaneous tissue at outer surface 30 of vessel wall 10. Using biocompatible materials to fabricate implantable monitoring device 312 and/or the coating layers on implantable monitoring device 312 may promote hemostasis and/or tissue in-growth. Additionally, one or more layers of a material, such as collagen, may be applied to radially inner surface 314 and/or a radially outer surface 316 of implantable monitoring device 312 to promote tissue in-growth and/or wound closure.

In one embodiment, implantable monitoring device 312 contains active or passive circuitry with electrical functionality. For example, implantable monitoring device 312 may contain a power source, such as a battery, an antenna, and/or an integrated circuit chip. Such circuitry processes, stabilizes, and/or boosts signals received from sensing elements in implantable monitoring device 312.

As shown in FIG. 19, implantable monitoring device 312 includes a hinge portion 320 to facilitate fitting implantable monitoring device 312 about vessel wall 10 and/or to facilitate obtaining accurate measurement readings, as described herein. In alternative embodiments, implantable monitoring device 312 includes any suitable expansion mechanism known to those skilled in the art and guided by the teachings herein provided that facilitates movement, such as expansion and/or contraction, of implantable monitoring device 312 to properly fit about the vessel.

In an alternative embodiment, implantable monitoring device 312 is fabricated at least partially from a material having shape memory properties. Suitable materials include, without limitation, Nitinol and other known shape memory alloys (SMA) having properties that develop a shape memory effect (SME), which allows the material to return to an initial configuration after a force applied to the material to shape, stretch, compress and/or deform the material is removed. In a further embodiment, implantable monitoring device 312 is fabricated from a thermally treated metal alloy (TMA) including, without limitation, nickel titanium, beta titanium, copper nickel titanium and any combination thereof. In an alternative embodiment, implantable monitoring device 312 is fabricated at least partially from a suitable polymeric material, such as a polyurethane material. It should be apparent to those skilled in the art and guided by the teachings herein provided that implantable monitoring device 312 may be made or fabricated using any suitable biocompatible material preferably, but not necessarily, having suitable shape memory properties.

In an exemplary embodiment, one or more sensors, such as load cells 340, are operatively coupled, directly or indirectly, to or incorporated within implantable monitoring device 312. Referring further to FIG. 19, in one embodiment, one or more load cells 340 are operatively coupled to implantable monitoring device 312 and configured to sense one or more physical, chemical, and/or physiological parameters or variables including, without limitation, a blood pressure within the vessel. Implantable monitoring device 312 may include additional sensors including, without limitation, a pressure sensor, an optical sensor, a biochemical sensor, a protein sensor, a motion sensor (e.g., an accelerometer or a gyroscope), a temperature sensor, a chemical sensor (e.g., a pH sensor), or a genetic sensor, for example.

In one embodiment, load cell 340 is coupled to implantable monitoring device 312 using a suitable biocompatible material. In one embodiment, load cell 340 is coupled to implantable monitoring device 312 using a suitable biocompatible adhesive including, without limitation, an acrylic-based adhesive, such as cyanoacrylate, an epoxy-based adhesive, a polyurethane-based adhesive, and/or a silicon-based adhesive, such as organopolysiloxane. Additionally or alternatively, load cell 340 is coupled to implantable monitoring device 312 using a suitable mechanism and/or process known to those skilled in the art and guided by the teachings herein provided including, without limitation, a chemical bonding process, a heat bonding process, a soldering process, a suturing process using a non-absorbable suture, or an outer packaging material. In a particular embodiment, the outer packaging material is chemically treated with a suitable heparin-bonded ePTFE material, a drug eluting material that inhibits cellular overgrowth, such as Tacrolimus or Sirolimus, or another suitable anti-metabolite to provide an anti-thrombotic outer surface.

Load cell 340 is coupled to or integrated with implantable monitoring device 312 during or after fabrication of implantable monitoring device 312. In one embodiment, load cell 340 is fabricated using a suitable microelectromechanical systems (MEMS) technology, such as described above in reference to sensor 40.

In one embodiment, load cell 340 is coated with at least one biocompatible material including, without limitation, one or more suitable biocompatible polymers such as a slow release polymer impregnated with an anti-metabolite inhibiting in-tissue growth. In a particular embodiment, a first portion of load cell 340 is coated with a drug eluting material that prohibits in-tissue growth on load cell 340. A second portion of load cell 340 is coated with a material promoting the in-tissue growth on load cell 340 to affix load cell 340 to vessel wall 10 for long term use.

In one embodiment, each sensor, including each load cell 340, is operatively coupled, such as in signal communication, with a RFID/magnetic/antenna component 342 that is operatively coupled, such as in signal communication, with an external receiver, such as described above.

Referring again to FIG. 20, in one embodiment, implantable monitoring device 312 is urged towards and retained in an expanded configuration by a suitable surgical instrument 350 such that implantable monitoring device 312 is positionable about vessel wall 10. With implantable monitoring device 312 properly positioned as desired, surgical instrument 350 releases implantable monitoring device 312 and implantable monitoring device 312 moves to a deployed configuration in which implantable monitoring device 312 is secured about vessel wall 10 at or near the closed incision.

In one embodiment, a method for treating cardiovascular disease includes generating, by one or more sensors, a signal representative of a physical, chemical, and/or physiological parameter or variable sensed or detected within the patient's vascular system. For example, in a particular embodiment, a sensor is positioned within a vessel of the patient's vascular system and is configured to generate a signal representative of a fluid pressure within the vessel to facilitate measuring and/or monitoring blood pressure within the patient's vascular system. The practicing physician is then able to control delivery of a pharmaceutical therapy based at least partially on the generated signal. The signal is communicated to a patient signaling device located at least partially externally to the patient. The signal is processed by the patient signaling device and instructive treatment signals are provided based on the processor output that will guide the patient and/or physician in determining a change in therapy.

In one embodiment, a method is provided for calibrating the measured pressure against external atmospheric pressure such that the adjusted pressure signal is based in part upon the signal sensor and the obtained atmospheric pressure.

In one embodiment, calibration of the device is initiated at initial manufacture and then at the time of implantation. The device coil is calibrated to a unique frequency signature of the device just prior to implantation with the reader set at atmospheric pressure based on a sea level height at which the procedure is taking place. Once zeroed and deployed, the sensor can then be recalibrated periodically by comparative measurement with standard blood pressure cuff readings. The recalibration of the device can also be performed by ultrasound interrogation. The piezoelectric signal and frequency shift generated by the deflection membrane as a prescribed set ultrasound frequency change can be used to determine a degree of membrane damping occurring as a result of cellular and non-cellular deposition.

In one embodiment, wherein the device includes a plurality of sensors, calibration includes placement of a reference sensor as one of the sensors. The reference sensor provides the ability to internally have affixed a reference point within the blood stream. The capacitance of the reference sensor will change only as a degree of cellular and non-cellular material deposit over time.

The reference sensor allows for calibration in addition to external calibration and accounts for drift in the signal over time based on a change in the materials as they are infiltrated and changed over time.

The multiple sensors also provide the ability to have more than one type of sensor, and up to six sensors in certain embodiments on the device of the same type, and between two and four sensors in alternative embodiments, which allows for summation of the signal or summation to the pressure points being derived. The summation allows averaging of the signal. The averaging of the signal allows for a more even distribution of the data set and increased confidence in the accuracy of the data.

The above-described vascular closure device allows for measuring and/or monitoring physical, chemical, and/or physiological parameters or variables within a patient's vascular system in addition to sealing an opening defined within a vessel at a puncture site. More specifically, a sensor coupled to or integrated with the vascular closure device allows a practicing physician to obtain diagnostic data, such as data representative of physical, chemical, and/or physiological variables, that may be useful to the practicing physician to assist in management of patient care after the procedure. The practicing physician may utilize data, such as blood pressure data, temperature data, blood chemical data, blood osmolar data, and/or cellular count data, to assist in directing patient care and making decisions regarding administration of medication, for example. The signal may also be integrated as a physiologic adjunct to anatomic measurements thereby creating an equivalent of real time functional and anatomic monitoring. This combination of physiologic and anatomic measurements can occur with the use of computer axial tomographic (CAT) scans, magnetic resonant imaging (MRI) scans and fluoroscopic dye examinations of the human body with special emphasis on the human vascular system.

Exemplary embodiments of a method and apparatus for measuring and/or monitoring physical, chemical, and/or physiological parameters or variables within a patient's vascular system in addition to sealing an opening at a puncture site are described above in detail. The method and apparatus are not limited to the specific embodiments described herein, but rather, steps of the method and/or components of the apparatus may be utilized independently and separately from other steps and/or components described herein. Further, the described method steps and/or apparatus components can also be defined in, or used in combination with, other methods and/or apparatus, and are not limited to practice with only the method and apparatus as described herein.

While the invention has been described in terms of various specific embodiments, those skilled in the art will recognize that the invention can be practiced with modification within the spirit and scope of the claims.

1. A vascular closure device comprising:
a first retainer positioned on an internal surface of a vessel wall defining a passage through a vessel;
a second retainer coupled to said first retainer and positioned on an outer surface of the vessel wall to seal an opening defined through the vessel wall; and
a sensor coupled to said first retainer and configured to sense at least one of a physical, chemical, and physiological parameter within the passage.

2. A vascular closure device in accordance with claim 1 further comprising an anchoring member positioned within the opening and coupling said first retainer to said second retainer.

3. A vascular closure device in accordance with claim 1 wherein said sensor is coupled to a radially inner surface of said first retainer.

4. (canceled)

5. (canceled)

6. A vascular closure device in accordance with claim 1 wherein said sensor is integrated with said first retainer.

7. A vascular closure device in accordance with claim 6 wherein, upon dissolution of said first retainer, said sensor is coupled to the internal surface of the vessel wall.

8. A vascular closure device in accordance with claim 1 wherein said sensor comprises a capacitor inductor circuit arranged in a parallel configuration and forming an LC tank circuit, said LC tank circuit having a resonating frequency emitted through the vessel and deciphered through an external device to generate an output representative of a parameter within the passage.

9. A vascular closure device in accordance with claim 8 wherein said sensor is fabricated utilizing a microelectromechanical systems (MEMS) technology.

10. A vascular closure device in accordance with claim 1 wherein said sensor comprises one of a capacitive pressure sensing device, a piezoelectric pressure sensing device and a piezoresistive pressure sensing device.

11. A vascular closure device in accordance with claim 1 wherein with said vascular closure device coupled to the vessel wall at a puncture site, said sensor attaching to the vessel wall.

12. A vascular closure device in accordance with claim 1 wherein at least a portion of said sensor is coated with at least one biocompatible material.

13. A vascular closure device in accordance with claim 12 wherein said at least a portion of said sensor is coated with at least one biocompatible polymer.

14. A vascular closure device in accordance with claim 12 wherein said at least one biocompatible material comprises a slow release polymer impregnated with an anti-metabolite inhibiting in-tissue growth, a drug eluting material that prohibits in-tissue growth on said sensor, and a material promoting in-tissue growth on said sensor to facilitate attaching said sensor to the vessel wall.

15. A vascular closure device in accordance with claim 1 wherein a first portion of said sensor is coated with a material prohibiting in-tissue growth on said sensor and a second portion of said sensor is coated with a material promoting the in-tissue growth on said sensor to facilitate attaching said sensor to the vessel wall.

16. A vascular closure device in accordance with claim 1 further comprising an anchoring device coupling said first retainer to said second retainer, at least one of said first retainer, said second retainer, and said anchoring device comprising a non-degradable biocompatible material.

17. A vascular closure device in accordance with claim 1 further comprising a patch applied to an outer skin surface of the patient, said patch in electrical communication with said sensor.

18. A vascular closure device comprising:
an inner retainer positioned on an internal surface of a vessel wall defining a passage through a vessel, said inner retainer comprising a wireless sensor configured to sense at least one of a physical, chemical, and physiological parameter within the passage; and
an outer retainer coupled to said inner retainer and positioned on an outer surface of the vessel wall to seal an opening defined through the vessel wall.

19. A vascular closure device in accordance with claim 18 wherein said inner retainer comprises:
an electrically conducting surface patterned in a void formed in a radially inner surface of said inner retainer; and
a plate coupled to said radially inner surface to enclose said void and form a hermetically sealed cavity, an inductor component and a capacitor plate patterned on said plate, said electrically conducting surface, said inductor, and said capacitor plate operatively coupled to form an LC tank circuit.

20. A vascular closure device in accordance with claim 19 wherein said inductor and said capacitor plate are electrically coupled in a parallel circuit.

21. A vascular closure device in accordance with claim 19 wherein said plate is configured to deform when a pressure is applied to said plate, the deformation producing a change in capacitance that causes a change in a resonant frequency of the LC tank circuit.

22. A vascular closure device in accordance with claim 19 wherein a resonant frequency of the LC tank circuit is monitored remotely with an external device.

23. A method for treating cardiovascular disease, said method comprising:
positioning a sensor within a passage defined by a vessel wall, the sensor configured to generate a signal representative of at least one of a physical, chemical, and physiological parameter within a vascular system of a patient;
communicating the signal to a patient signaling device located at least partially externally to the patient; and
processing the signal within the patient signaling device to generate at least one instructive treatment signal to facilitate determining a pharmaceutical therapy.

24. A method in accordance with claim 23 further comprising controlling delivery of the pharmaceutical therapy based at least partially on the processed signal.

25. A vascular closure device comprising at least one sensor configured for measuring at least one of a physical, chemical, and physiological parameter of a patient.

26. A vascular closure device in accordance with claim 25 wherein said at least one of a physical, chemical, and physiological parameter includes at least one of a pressure, a temperature, and a cardiac output.

27. A method for measuring at least one of a physical, chemical, and physiological parameter of a patient comprising placing a permanent implant with respect to a vessel wall.

28. A device for measuring at least one of a physical, chemical, and physiological parameter of a patient comprising a sensor permanently implanted in proximity to a vessel wall.

29. A device in accordance with claim 28 wherein said sensor is coupled to one of an outer surface of said vessel wall and an inner surface of said vessel wall.

30. A single-piece flexible vascular closure device comprising:
a first portion positioned within a vessel at an interface of said vascular closure device and a flow of blood through the vessel;
a second portion transitioning into said first portion, at a transition area, at least one of said first portion and said second portion forming a groove configured to receive a portion of a vessel wall defining an opening through the vessel wall, said second portion positioned on an outer surface of the vessel wall to seal the opening; and
a sensor operatively coupled to one of said inner portion and said outer portion, said sensor configured to sense at least one of a physical, chemical, and physiological parameter within the vessel,

31. A single-piece flexible vascular closure device in accordance with claim 30 wherein said sensor is one of directly coupled to said first portion and integrated with said first portion.

32. An implantable monitoring device positionable externally about a vessel, said implantable monitoring device comprising at least one of a load cell and a sensor configured to sense at least one of a physical, chemical, and physiological parameter within the vessel.

33. An implantable monitoring device in accordance with claim 32 further comprising a hinge portion to facilitate fitting said implantable monitoring device about a vessel wall.

34. An implantable monitoring device in accordance with claim 32 wherein at least a portion of said implantable monitoring device is fabricated from at least one of a material having shape memory properties and a polymeric material.

35. An implantable monitoring device in accordance with claim 32 wherein said sensor is configured to sense at least one of a physical, chemical, and/or physiological parameter within the vessel.

36. An implantable monitoring device in accordance with claim 35 wherein said sensor comprises one a pressure sensor, an optical sensor, a biochemical sensor, a protein sensor, a motion sensor, an accelerometer, a gyroscope, a temperature sensor, a chemical sensor, a pH sensor, and a genetic sensor.

37. An implantable monitoring device in accordance with claim 32 wherein said at least one of a load cell and a sensor is fabricated using a microelectromechanical systems (MEMS) technology.

38. An implantable monitoring device in accordance with claim 32 further comprising a RFID/magnetic/antenna component operatively coupled with said at least one of a load cell and a sensor, said RFID/magnetic/antenna component operatively coupled to an external receiver.

39. An implantable device comprising:
a first material defining a void on a first surface, and an electrically conducting surface patterned in said first surface within said void; and
a plate coupled to said first surface to enclose said void, forming a hermetically sealed cavity, said plate patterned with electrically functional components, said implantable device having pressure sensing capabilities.
